(19) United States
(12) Patent Application Publication
(10) Pub. No.: US 2013/0071340 A1
(43) Pub. Date: Mar. 21, 2013

(54) POLYOL PARTIAL ESTERS FOR USE IN COSMETICS

(71) Applicants: Hans Henning Wenk, Muelheim an der Ruhr (DE); Juergen Meyer, Essen (DE); Oliver Springer, Wesel (DE); Mike Farwick, Essen (DE); Klaus Jenni, Essen (DE); Wolfgang Berkels, Bottrop (DE); Frank Unger, Oberhausen (DE)

(72) Inventors: Hans Henning Wenk, Muelheim an der Ruhr (DE); Juergen Meyer, Essen (DE); Oliver Springer, Wesel (DE); Mike Farwick, Essen (DE); Klaus Jenni, Essen (DE); Wolfgang Berkels, Bottrop (DE); Frank Unger, Oberhausen (DE)

(73) Assignee: EVONIK GOLDSCHMIDT GMBH, Essen (DE)

(21) Appl. No.: 13/701,358

(22) PCT Filed: Apr. 27, 2011
(86) PCT No.: PCT/EP2011/056616

§ 371 Date: Nov. 30, 2012

(30) Foreign Application Priority Data
May 31, 2010 (DE) 10 2010 029 499.3

(51) Int. Cl. A61K 8/92; A61Q 17/04; C11C 3/02; A61K 8/81; A61K 8/85

(52) U.S. Cl. CPC A61K 8/92; C11C 3/025; A61Q 17/04
USPC 424/59; 554/227; 514/786

(57) ABSTRACT

The invention relates to polyol partial esters, cosmetic and pharmaceutical formulations containing the polyol partial esters according to the invention, and to the use of the polyol partial esters according to the invention for cosmetic and pharmaceutical purposes.

### FIELD OF THE INVENTION

The invention relates to polyol partial esters, cosmetic and pharmaceutical formulations containing the polyol partial esters according to the invention and the use of the polyol partial esters according to the invention for particular cosmetic purposes.

### PRIOR ART

Cosmetic products for skin care applications usually consist of emulsions that contain oily substances. In addition, cosmetic emulsions generally contain cosmetic active substances, for producing special effects for protection and for regeneration of the skin.

Cosmetic oily substances should preferably provide a caring, but non-sticky or oily sensation on the skin. The oily substances contained in emulsions therefore also have a decisive influence on good distribution of cosmetic emulsions and rapid absorption.

Silicone oils, especially cyclomethicones, are widely used cosmetic oily substances, which bring about good distribution of cosmetic emulsions. Owing to its high volatility, cyclomethicone evaporates from the skin very quickly without causing a sticky sensation.

One drawback in using silicone oils in cosmetics is for example their inadequate biodegradability. However, silicone oils also have definite weaknesses with regard to their compatibility with cosmetic active substances. Owing to their strongly hydrophobic character, silicone oils are in particular barely able to dissolve active substances of a polar character. Furthermore, silicone oils are virtually unable to promote the penetration of cosmetic active substances into the skin.

Polyol partial esters, for example partial esters of glycerol with fatty acids, are known per se in the cosmetic industry.

For example, DE 19631004 and DE 19543696 describe cosmetic preparations comprising one or more substances selected from the group of the monoglycerol-monocarboxylic acid monoesters, diglycerol-monocarboxylic acid monoesters, triglycerol-monocarboxylic acid monoesters, monoglycerol-dicarboxylic acid monoesters, diglycerol-dicarboxylic acid monoesters and triglycerol-dicarboxylic acid monoesters. Esters mentioned therein are glyceryl monocaprylate GMCy, glyceryl monocaprate GMC, glyceryl monostearate GMS, glyceryl monoundecylate GMU, diglyceryl monocaprate DMC, triglyceryl monolaurate TML, triglyceryl monomyristate TMM. The proportion of these esters is 0.1 to 10 wt % in the formulations in DE 19631004 or DE 19543696.

A method of producing carboxylic acid esters is described in DE 3818293.

Difatty acid diglyceryl esters with defined hydrophilicity, which produce improved stability of W/O emulsions relative to diglyceryl monoesters or higher polyglyceryl esters, are also disclosed therein.

EP 1762216 discloses liquid makeup removers with nonionic surfactants and glycerol partial esters of a C6 to C12 fatty acid, wherein the total proportion of mono- and diesters is more than 50% and the weight ratio of mono- to diesters is 4 or less.

EP 1800650 describes hydroxyesters from the esterification of a polyol with C4-C16 acids, with a high proportion of monoesters.

EP 531684 describes combinations of glyceryl diesters with a total of 13 to 20 carbon atoms with polyols and surfactants for cleansing agents. The amount of surfactant in the cleansing agent is 10 to 50% and is selected to be at least twice as high as the amount of glyceryl diester.

EP 522624 discloses glyceryl caprylate and glyceryl caprate as preferred emulsifiers for special lipstick W/O emulsions.

U.S. Pat. No. 6,265,372 describes combinations of glyceryl diesters with C3-C12 fatty acids with silicones and surfactants for cleansing agents, wherein the amount of surfactant is at least as high as the amount of glyceryl diester.

DE 102008013023 describes a method of producing octanoic acid glyceryl esters with a molar ratio of glycerol to octanoic acid in the range from 1 to 1, to 1 to 0.45.

It is known that glycerol partial esters with a high proportion of monoglyceride are very suitable for promoting the penetration of cosmetic active substances into the skin. Typical products providing such action are for example AKOLINE products from the company Karlshamns, The AKOLINE products are partial esters of glycerol with caprylic/capric acid with a proportion of monoglyceride 50%. A similar product offered is for example IMWITOR 308 from the company Sasol. This is a partial ester of glycerol with octanoic acid with a proportion of monoglyceride >80%.

Both the product IMWITOR 308 (melting point 30-34° C.) and the AKOLINE products (melting point 25-28° C.) are solid or pasty crystalline masses at room temperature. These solid glycerol partial esters with a proportion of monoglyceride >50% therefore cannot be used as cosmetic oils with good distribution properties and a light sensation on the skin.

Furthermore, it is also not possible to use these glycerol partial esters with high monoglyceride content as solvents for active substances or UV filters, as they are not liquid at room temperature.

The high proportion of monoglyceride in these products of the prior art also leads to poor compatibility with nonpolar oils such as mineral oil, which are used in many cosmetic formulations.

Owing to the highly polar character, the glycerol partial esters of the prior art are therefore even described as emulsifiers and not as oily substances, cf. the product documentation for ALKOLINE and Imwitor 308, as well as EP0522624.

The problem to be solved by the invention was therefore to overcome at least one drawback of the prior art, and in particular provide oily substances that ensure good bioavailability of active substances in the skin.

### DESCRIPTION OF THE INVENTION

It was found, surprisingly, that the polyol partial esters described hereunder are able to solve the technical problem of the invention.

The present invention therefore relates to polyol partial esters as described in claim 1.

The invention further relates to cosmetic and pharmaceutical formulations containing polyol partial esters according to the invention.

The present invention further relates to the use of the polyol partial esters according to the invention as solubilizers for cosmetic active substances, and as penetration enhancers of these active substances for increasing the bioavailability.

The invention further relates to the use of the polyol partial esters according to the invention for solvating UV filters, in particular in cosmetic formulations.

The polyol partial esters according to the invention are liquid at room temperature and can be used as oily substances in cosmetic formulations.

When used in cosmetic formulations, the polyol partial esters according to the invention are characterized in particular by a non-oily, non-sticky, dry sensation on the skin, which makes them interesting as nonvolatile substitutes for cyclomethicones.

Another advantage of the polyol partial esters according to the invention is that they can be used as oily substances in cosmetic formulations with active substances and can provide better efficacy of the active substances used, as they bring about improved penetration of the active substances into the skin. A great advantage over known penetration intensifiers such as glyceryl monocaprylate (solid product) is the very good compatibility of the polyol partial esters with typical nonpolar cosmetic oils, e.g. mineral oil.

Furthermore, the polyol partial esters according to the invention are characterized by very good UP filter solubility, which makes them preferred emollients for preparing cosmetic sunscreen formulations (see the practical examples).

Yet another advantage of the polyol partial esters according to the invention is that they spread well on the skin.

A further advantage is that the polyol partial esters according to the invention can in many cases be produced completely from renewable raw materials.

Another advantage of the polyol partial esters according to the invention is that they are miscible with other oily phases, without any need for melting or heating.

Yet another advantage of the invention is that the polyol partial esters provide greater sun protection for UV filters, than with usual solvents.

Polyol partial esters according to the invention are esterification products of at least one polyol, selected from polyols with 3 to 6 carbon atoms containing 2 to 6, preferably 3 to 6, OH groups, with at least one carboxylic acid containing 5 to 18, preferably 6 to 12, especially preferably 8 to 10, carbon atoms,

with the proviso that the molar ratio of the OH groups in the polyol to the acyl groups of the carboxylic acids in the polyol partial ester is between 1:0.90 and 1:0.35, preferably between 1:0A3 and 1:0.40 and especially preferably between 1:0,70 and 1:0.45.

The term “polyol partial ester” means, in connection with the present invention, mixtures of various polyol esters, which differ by their degree of esterification; thus, for example, a glyceryl partial ester according to the invention can contain at least two of the glyceryl esters, selected from glyceryl monoester (or monoglyceride), glyceryl diester (or diglyceride) and glyceryl triester (or triglyceride); the full ester is in this context explicitly included as a possible constituent of the mixture. All percentages stated (%) are percentages by weight, unless stated otherwise.

Polyol partial esters preferred according to the invention are characterized in that they have a melting point below 22° C., in particular below 20° C., at 1 bar pressure.

Preferred polyols are sugars, such as pentoses and hexoses, and sugar alcohols, and anhydrides thereof, in particular those selected from the group comprising, preferably consisting of, sorbitol, mannitol, xylitol, erythritol, arabitol, sorbitan and simple polyols such as in particular pentaerythritol, trimethylolmethane, trimethylolethane, trimethylolpropane, 1,2,4-butanetriol, 1,2-propanediol, 1,3-propanediol and glycerol, wherein glycerol in this context is especially preferred according to the invention. Unsubstituted, linear or branched carboxylic acids are used in particular for obtaining the polyol partial esters. They are, according to the invention, preferably saturated, although it may also be advantageous for certain applications to use aromatic carboxylic acids, in particular benzoic acid.

Carboxylic acids preferably used for obtaining the polyol partial esters are in particular saturated carboxylic acids not containing any heteroatoms. In particular this group comprises, preferably consists of, neopentanoic acid, isoamylic acid, pentanoic acid, n-hexanoic acid, 2-ethylbutanoic acid, cyclohexanecarboxylic acid, n-octanoic acid, 2-ethylhexanoic acid, isononanoic acid, 3,5,5-trimethylhexanoic acid, n-decanoic acid, iso-decanoic acid, lauric acid and 2-butyloctanoic acid, especially preferably 2-ethylbutanoic acid, 2-ethylhexanoic acid, n-octanoic acid and n-decanoic acid, in particular n-octanoic acid and n-decanoic acid.

It is preferable, according to the invention, that per mol of OH group contained in the polyol, in total 0.45 to 0.70 mol of the OH groups esterified with acyl groups of the carboxylic acid are present in the polyol partial ester.

In particular, those polyol partial esters are preferred for which each individual polyol ester of a certain degree of esterification (for example monoester, diester, triester) contained in the polyol partial ester accounts for not more than 80 wt %, preferably not more than 70 wt % and especially preferably not more than 60 wt % relative to the total polyol partial ester. In particular, those polyol partial esters are preferred for which the content of monoester is less than 60 wt %, in particular less than 50 wt %, especially preferably less than 45 wt %, relative to the total polyol partial ester.

The polyol partial esters according to the invention are, moreover, obtainable by thermal methods known per se by a person skilled in the art, as described for example in DE102008013023, WO 9811179, EP 407959, Ullmann's Encyclopedia of Industrial Chemistry, 6th ed, 2002, chapter “Esters, Organic” or enzymatic methods as described in U.S. Pat. No. 6,613,551 and U. T. Bornscheuer in “Enzyme and Microbial Technology”, 17, 578-586, 1995.

Accordingly, the polyol and the carboxylic acid are reacted together in conditions for esterification reactions known per se by a person skilled in the art, optionally in the presence of a catalyst. In particular, the esterification is carried out with removal of water from the reaction mixture. The thermal process for obtaining the polyol partial esters is preferably carried out at 120-260° C., especially preferably 160-250° C. In the case of an enzymatically catalyzed esterification reaction, the process temperature is to be adjusted correspondingly to a range from 20 to 80° C., preferably 30-60° C. The progress of the reaction can for example be monitored on the basis of the acid number, for example by the method in DIN53402 or DIN EN ISO 2114, of the product, so that it is preferable to carry out the reaction until the desired acid number is reached.

It is apparent to a person skilled in the art that mixtures of polyols and/or mixtures of the carboxylic acids can be used for preparing the polyol partial esters.

In this context, a preferred embodiment is characterized in that for obtaining the polyol partial esters, a mixture of n-octanoic acid and n-decanoic acid is used in a weight ratio from 40:80 to 20:60, preferably from 50:70 to 30:50, and in particular from 55:65 to 35:47, wherein it is especially preferable according to the invention that per mol of OH group contained in the polyol, a total of 0.45 to 0.70 mol, preferably 048 to 0.52 mol of acyl groups of octanoic acid and decanoic acid are esterified in the polyol partial ester. In this context, the polyol used is in particular glycerol.

Preferred partial esters are obtainable from
1 mol glycerol with 1 mol lauric acid and 1.5 mol benzoic acid
1 mol glycerol with 1 mol octanoic acid and 1 mol benzoic acid
1 mol glycerol with 1 mol octanoic acid and 1.5 mol benzoic acid
1 mol glycerol with 2 mol C8/C10 fatty acid
1 mol glycerol with 1.5 mol C8/C10 fatty acid and 1 mol benzoic acid
1 mol glycerol with 1 mol benzoic acid and 2 mol C8/C10 fatty acid
1 mol glycerol with 1 mol benzoic acid and 1 mol C8/C10 fatty acid
1 mol glycerol with 1 mol benzoic acid and 1 mol isononanoic acid
1 mol glycerol with 0.8 mol benzoic acid and 0.8 mol C8/C10 fatty acid
1 mol glycerol with 1 mol benzoic acid and 1 mol 2-bropylheptanoic acid
1 mol glycerol with 2 mol isononanoic acid
1 mol glycerol with 0.5 mol benzoic acid and 1.5 mol C8/C10 fatty acid
1 mol glycerol with 1.5 mol benzoic acid and 0.5 mol C8/10 fatty acid
1 mol glycerol with 0.25 mol benzoic acid and 1.75 mol C8/C10 fatty acid
1 mol glycerol with 0.50 mol benzoic acid and 1.50 mol isononanoic acid
1 mol glycerol with 0.67 mol lauric acid and 133 mol C8/10 fatty acid
1 mol trmethylolprobane with 1 mol benzoic acid and 1 mol C8/C10 fatty acid
1 mol glycerol with 2 mol isooctancic acid
1 mol trimethylolpropahe with 2 mol C8/C10 fatty acid
1 mol glycerol with 2 mol isodecanoic acid
1 mol glycerol with 2 mol neodecanoic acid
1 mol pentaerythritol with 3 mol C8/C10 fatty acid
1 mol pentaerythritol with 1.5 mol benzoic acid and 1.5 mol C8/C10 fatty acid
1 mol glycerol with 0.5 mol isononanoic acid and 1.5 mol benzoic acid
1 mol glycerol with 2 mol 2-ethylbutyric acid
1 mol glycerol with 1 mol 2-ethylbutyric acid and 1 mol benzoic acid
1 mol glycerol with 2.5 mol C8/C10 fatty acid
1 mol glycerol with 1.5 mol C8/C10 fatty acid
1 mol glycerol with 2.5 mol isononanoic acid
1 mol glycerol with 1.5 mol 2-ethylbutyric acid
1 mol glycerol with 2.5 mol 2-ethylbutyric acid
1 mol glycerol with 1.5 mol isononanoic acid
1 mol glycerol with 2 mol iso-C6-acid (Versatic 6)
1 mol glycerol with 2 mol neopentanoic acid
1 mol glycerol with 2 mol pentanoic acid
1 mol glycerol with 2 mol hexananoic acid
1 mol 1,2-bentanediol with 1 mol C8/C10 fatty acid
1 mol 1,2-pentanediol with 1 mol isononanoic acid
1 mol 1,2-octanediol with 1 mol C8/10 fatty acid
1 mol 1,2-octanediol with 1 mol 2-ethylbutanoic acid
1 mol diglycerol with 3 mol 2-ethylbutanoic acid
1 mol diglycerol with 3 mol C8/10 fatty acid

Especially preferred partial esters are obtainable from:
1 mol glycerol with 2 mol C8/C10 fatty acid
1 mol glycerol with 1.5 mol C8/C10 fatty acid
1 mol glycerol with 2.5 mol 2-ethylbutyric acid
1 mol glycerol with 2 mol isooctanoic acid
1 mol glycerol with 2 mol 2-ethylbutyric acid
1 mol glycerol with 2.5 mol C8/C10 fatty acid
   as is also shown in the following Table 1.

The invention further relates to cosmetic and pharmaceutical formulations containing polyol partial esters according to the invention.

As polyol partial esters according to the invention promote penetration of low-molecular substances, in particular cosmetic and pharmaceutical active ingredients as listed below for example, into the upper layers of the skin, preferred formulations contain at least one cosmetic and/or pharmaceutical active substance.

The term active substance generally includes active substances that exert a desired physiological or physical effect on the human or animal body or in the case of active pharmaceutical ingredients serve for the prevention or treatment of clinical conditions or deficiency symptoms.

Thus, this also includes herbal extracts, vitamins, antibiotics and other components with medicinal action.

The following are to be understood, for example, as said active substances: tocopherol, tocopherol acetate, tocopherol palmitate, ascorbic acid, deoxyribonucleic acid, coenzyme Q10, retinol, bisabolol, allantoin, phytanetriol, panthenol, AHA acids, amino acids, hyaluronic acid, alpha-hydroxy acids, polyglutamic acid, creatine (and creatine derivatives) , guanidine (and guanidine derivatives), ceramides, phytosphingosine (and phytosphingosine derivatives), sphingosine (and sphingosine derivatives), pseudoceramides, sphingolipids, essential oils, peptides and oligopeptides, protein hydrolysates, plant extracts and vitamin complexes, and benzyl peroxide, niacinamide hydroxybenzoate, nicotinaldehyde, retinol acid, salicylic acid, citronellic acid, xanthine compounds such as caffeine, theophylline, theobromine and aminophylline, carnitine, carnosine, kojic acid, arbutin, vitamin C, hydroquinone.

A preferred formulation according to the invention is characterized in that it contains
a) 0.1-60 wt %, preferably 1 to 25 wt % and especially preferably 3 to 15 wt % of polyol partial esters according to the invention,
(b) 0.001-15 wt %, preferably 0.05-10 wt %, especially preferably 0.1-5 wt % of at least one cosmetic and/or pharmaceutical active substance,
(c) 0.1-20 wt %, preferably 0.2-10 wt %, especially preferably 0.5-6 wt % of emulsifiers and/or coemulsifiers,
(d) 0.1-40 wt %, preferably 0.2-20 wt %, especially preferably 0.5-15 wt % of other oily substances,
(e) 0-99 wt %, preferably 10-95 wt %, especially preferably 30-75 wt % of water and optionally usual excipients and additives,
   wherein the percentages by weight refer to the total formulation, and the percentages by weight of components (a), (b), (c), (d) and (e) preferably add up to 100 wt %.

Active substances preferably contained are selected from the group consisting of: tocopherol, tocopherol acetate, tocopherol palmitate, ascorbic acid, coenzyme Q10, retinol, bisabolol, allantoin, panthenol, amino acids, hyaluronic acid, alpha-hydroxy acids, polygiutamic acid, creatine (and creatine derivatives), guanidine (and guanidine derivatives), ceramides, phytosphingosine (and phytosphingosine derivatives), sphingosine (and sphingosine derivatives), pseudoceramides, sphingolipids, essential oils, peptides and oligopeptides, protein hydrolysates, plant extracts and vitamin complexes, niacinamide, retinol acid, salicylic acid, compounds such as caffeine, carnitine, carnosine, kojic acid and arbutin.

The present invention further relates to cosmetic sunscreen formulations containing polyol partial esters according to the invention.

The term “cosmetic sunscreen formulation” means, in connection with the present invention, a cosmetic composition for topical application, which owing to its ingredients is obviously suitable for reducing the radiation incident on the surface, for a surface that is exposed to UV radiation. Said compositions include in particular those that contain at least one of the UV filter substances described hereunder.

Cosmetic sunscreen formulations according to the invention preferably contain UV filters. It is possible to use, as UV filters, for example organic substances that are able to absorb ultraviolet rays and reemit the absorbed energy in the form of longer-wave radiation, e.g. heat. UVB filters can be oil-soluble or water-soluble, As oil-soluble UVB filters, we may mention for example:
3-benzylidenecamphor and derivatives thereof, e.g. 3-(4-methylbenzylidene)camphor,
4-aminobenzoic acid derivatives, for example
4-(dimethylamino)benzoic acid-2-ethylhexyl ester,
4-(dimethylamino)benzoic acid-2-ethylhexyl ester and
4-(dimethylamino)benzoic acid amyl ester,
esters of cinnamic acid, for example 4-methoxycinnamic acid-2-ethylhexyl ester, 4-methoxy cinnamic acid isopentyl ester, 2-cyano-3-phenyl-cinnamic acid-2-ethylhexyl ester (octocrylenes),
esters of salicylic acid, for example salicylic acid-2-ethylhexyl ester, salicylic acid-4-isopropylbenzyl ester, salicylic acid homomenthyl ester,
derivatives of benzophenone, for example 2-hydroxy-4-methoxybenzophenone,
2-hydroxy-4-methoxy-4′-methylbenzophenone,
2,2′-dihydroxy-4-methoxybenzophenone,
esters of benzalmalonic acid, for example 4-methoxybenzalmalonic acid di-2-ethylhexyl ester,
triazine derivatives, for example 2,4,6-trianilino-(p-carbo-2′-ethyl-1′-hexyloxy)-1,3,5-triazine, octyltriazone and those described in EP 1180359 and DE 2004/027475,
propane-1,3-diones, for example 1-(4-tert-butylphenyl)-3-(4′-methoxyphenyl)propane-1,3-dione.

The following may come into consideration as water-soluble UVB filters:
2-phenylbenzimidazole-5-sulfonic acid and their alkali-metal, alkaline-earth, ammonium, alkylammonium,
alkanolammonium and glucammonium salts,
sulfonic acid derivatives of benzophenone, for example 2-hydroxy-4-methoxybenzophenone-5-sulfonic acid and salts thereof,
sulfonic acid derivatives of the 3-benzylidenecamphors, for example 4-(2-oxo-3-bornylidenemethyl)benzene sulfonic acid and 2-methyl-5-(2-oxo-3-bornylidene)sulfonic acid and salts thereof.

Derivatives of benzoylmethane may in particular come into consideration as typical OVA filters, for example 1-(4′-tert-butylphenyl)-3-(4′-methoxyphenyl)propane-1,3-dione or 1-phenyl-3-(4′-isopropylphenyl)propane-1,3-dione. The UV-A and UVB filters can of course also be used in mixtures.

In addition to the aforementioned soluble substances, insoluble pigments, namely finely dispersed metal oxides or salts, may also be considered for this purpose, for example titanium dioxide, zinc oxide, iron oxide, aluminum oxide, cerium oxide, zirconium oxide, silicates (talc), barium sulfate and zinc stearate. The particles should have an average diameter of less than 100 nm, e.g. between 5 and 50 nm and in particular between 15 and 30 nm. They can have a spherical shape, but it is also possible to use particles that have an ellipsoid shape or some other form different from spherical. A relatively new class of light protection filters comprises micronized organic pigments, for example 2,2′-methylene-bis-{6-(2H-benzotriazol-2-yl)-4-(1,1,3,3-tetramethylbutyl)-phenol} with a particle size of <200 nm, which is obtainable for example as 50% aqueous dispersion.

Other suitable UV filters are given in the review by P. Finkel in SÖFW-Journal 122, 543 (1996).

In addition to the two aforementioned groups of primary UV filters, it is also possible to use secondary light protective agents of the antioxidant type, which interrupt the photochemical reaction chain that is initiated when UV radiation penetrates into the skin.

In connection with the sunscreen formulations according to the invention, these preferably contain the lipophilic, hydrophobic UV filters, in particular triazine derivatives. Especially preferably, the UV filter substances 2-cyano-3-phenyl-cinnamic acid-2-ethylhexyl ester, 2,4-bis-{[4-(2-ethylhexyloxy)-2-hydroxy]-phenyl}-6-(4-methoxyphenyl)-1,3,5-triazine, dioctylbutylamidotriazone, 2-hydroxy-4-methoxybenzophenone, 2-hydroxy-4-methoxy-4′-methylbenzophenone, 2,2′-dihydroxy-4-methoxybenzophenone, 4-methoxybenzalmalonic acid di-2-ethylhexyl ester, 2,4,6-tris-[anilino-(p-carbo-2′-ethyl-1′-hexyloxy)]-1,3,5-triazine, 2,4-bis-[5,1(dimethylpropyl)benzoxazol-2-yl-(4-phenyl)-imino]-6-(2-ethylhexyl)-imino-1,3,5-triazine, 2,4-bis-{[4-(2-ethylhexyloxy)-2-hydroxy]-phenyl}-6-(4-methoxyphenyl)-1,3,5-triazine and 2-[4,6-bis(2,4-dimethylphenyl)-1,3,5-triazin-2-yl]-5-(octyloxy)phenol are used here as UVB filters.

Preferably 1-(4′-tert-butylphenyl)-3-(4′-methoxyphenyl)propane-1,3-dione, 1-phenyl-3-(4′-iso-oropylphenyl)-propane-1,3-dione are used as UVA filters.

Especially preferred UVA filters are 4-(tert-butyl)-4′-methoxydibenzoylmethane (CAS No. 70356-09-1), which is sold by Givaudan under the brand name Parsol® 1789 and by Merck under the trade name Eusolex® 9020, and hydroxybenzophenones according to DE 102004027475, especially preferably 2-(4′-diethylamino-2′-hydroxybenzoyl)-benzoic acid hexyl ester (also: aminobenzophenone), which is obtainable under the name Uvinul A Plus from the company BASF.

Other preferred UV filter substances are moreover so-called wideband filters, i.e. filter substances that absorb both UV-A and UV-B radiation, Within this group, 2,2-methylene-bis (6-(2H-benzotriazol-2-yl)-4-(1,1,3,3-tetramethylbutyl)-phenol, which is obtainable under the trade name Tinosorb® M from the company BASF and 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS No.: 155633-54-8) with the INCI name drometrizole trisiloxane, are preferably used.

The amount of the UV filters to be used is preferably 0.01-20%, preferably 0.05-15%, especially preferably 0.1-10% relative to the formulation.

The use of a combination of several different UV filters is preferred.

Another additional component that is preferably used in the sunscreen formulation according to the invention is the group of film-forming agents, to make the compositions more water-proof and therefore also increase the UV-protection performance. Film-forming agents that are preferably used are polyurethanes, dimethicones, copolyols, polyacrylates, PVP/VA copolymers (PVP=polyvinylpyrrolidone, VA=vinyl acetate), polyvinylpyrrolidone (PVP), polyvinylpyrrolidone copolymers, PVP/hexadecene copolymer or PVP/eicosene copolymer.

The cosmetic or pharmaceutical formulation according to the invention can for example contain at least one additional component, selected from the group of
emollients,
emulsifiers,
thickeners/viscosity regulators/stabilizers,
antioxidants,
substances with deodorant and antiperspirant action,
hydrotropic substances (or polyols),
solid substances and fillers,
nacreous additives,
insect repellents,
self-tanning agents,
preservatives,
conditioners,
perfumes,
colorants,
care additives,
overfatting agents,
solvents.

Substances that can be used as examples of representatives of the individual groups are known by a person skilled in the art and can be found for example in German application DR 102008001788.4. This patent application is incorporated hereby as reference and therefore forms part of the disclosure.

Regarding other optional components and the amounts of these components to be used, reference is expressly made to the relevant handbooks known by a person skilled in the art, e.g. K. Schrader, “Grundlagen und Rezepturen der Kosmetika” [Principles and recipes of cosmetics], 2nd edition, pages 329 to 341, Huthig Buch Verlag Heidelberg.

The amounts of the respective additives depend on the intended use.

Typical starting formulations for the respective uses are known from the prior art and are given for example in the brochures of the manufacturers of the respective bases and active substances. These existing formulations can as a rule be adopted unaltered. If necessary, however, the desired modifications for adapting and optimizing can be made without difficulty by simple tests.

Formulations according to the invention can for example find application in the form of an emulsion, a suspension, a solution, a cream, an ointment, a paste, a gel, an oil, a powder, an aerosol, a stick, a spray or a foam.

A preferred cosmetic sunscreen formulation according to the invention is characterized in that it contains
(a) 0.1-60 wt %, preferably 1 to 25 wt % and especially preferably 3 to 15 wt % of polyol partial esters according to the invention,
(b) 0.01-20 wt %, preferably 0.05-15 wt %, especially preferably 0.1-10 wt % of at least one UV filter substance,
(c) 0.1-20 wt %, preferably 0.2-10 wt %, especially preferably 0.5-6 wt % of emulsifiers and/or coemulsifiers,
(d) 0.1-40 wt %, preferably 0.2-20 wt %, especially preferably 0.5-15 wt % of other oily substances,
(e) 0-99 wt %, preferably 30-95 wt %, especially preferably 10-75 wt % water and optionally usual excipients and additives,
   wherein the percentages by weight refer to the total formulation, and the percentages by weight of components (a), (b), (c), (d) and (e) preferably add up to 100 wt %.

A further contribution to solving the problem mentioned at the beginning is made by a method of producing the cosmetic sunscreen formulation according to the invention comprising the process steps
(A) dissolving the organic CV filters in an oily phase containing, preferably consisting of, polyol partial esters according to the invention,
(B) providing an aqueous phase and combining the aqueous and oily phase,
(C) homogenizing and, in the case of a hot process, cooling.

A further contribution to solving the problem mentioned at the beginning is made by using the polyol partial esters according to the invention as solubilizers or solvents for at least one UV filter substance, in particular in cosmetic formulations.

Another contribution to solving the problem mentioned at the beginning is made by using the polyol partial esters according to the invention for increasing the photoprotective action of at least one UV filter substance, in particular in cosmetic formulations.

In this context the term “increasing the photoprotective action of at least one UV filter substance” means an increase compared to conventional emollients, in particular to ethylhexyl palmitate, dicaprylyl carbonate, diethylhexyl carbonate, caprylic/capric triglycerides, C12-15 alkyl benzoate, octyldodecanol, isopropyl palmitate, isopropyl myristate, butylene glycol dicaprylate/dicaprate and paraffinum liquidum: if a formulation with conventional emollient possesses a certain SPF (sun protection factor), then the increase of this given SPF is to be measured by the method described in the examples, compared to a formulation in which this conventional emollient has been replaced with polyol partial esters according to the invention.

Yet another contribution to solving the problem mentioned at the beginning is made by a cosmetic use of the polyol partial esters according to the invention as enhancers of the penetration of cosmetic active substances into the skin. In this context the term “penetration into the skin” means penetration of the active substance into the upper layers of the skin. Penetration of active substances into the vascularized layers of the skin, indicated by analytical detectability of the active substances in the receptor medium in the penetration tests described below, was not observed in a single case when using polyol partial esters according to the invention. Therefore the observed improved bioavailability of active substances is limited to a cosmetic use and not to a therapeutic-pharmaceutical use.

In the method of producing the cosmetic sunscreen formulation according to the invention, when using the polyol partial esters according to the invention as solubilizers or solvents for UV filters and in the cosmetic use of the polyol partial esters according to the invention for enhancing the penetration of cosmetic active substances into the skin and when using the polyol partial esters according to the invention for increasing the photoprotective action of at least one UV filter substance, preferably those polyol partial esters according to the invention are used that are described above as preferably described polyol partial esters.

In the method of producing the cosmetic sunscreen formulation according to the invention and when using the polyol partial esters according to the invention as solubilizers or solvents for UV filters and when using the polyol partial esters according to the invention for increasing the photoprotective action of at least one UV filter substance, preferably those UV filter substances are used that are contained in the cosmetic sunscreen formulations described above, in particular in the sunscreen formulations described-above as preferable.

In the cosmetic use of the polyol partial esters according to the invention for enhancing the penetration of cosmetic active substances into the skin, preferably those cosmetic active substances are used that are described above as active substances preferably contained in compositions according to the invention.

In the examples presented below, the present invention is described as examples, without limiting the invention, whose range of application follows from the complete description and the claims, to the embodiments mentioned in the examples.

### EXAMPLES

Examples of polyol partial esters according to the invention:

Distribution of chains of the precursor fatty acid used in the examples (C8/010 fatty acid):
C6: <2%
C8: 53-64%
C10: 36-47%
C12: <2%

### Example 1

139.9 g (1.5207 mol) of glycerol and 360.1 g (2.2852 mol) of precursor fatty acid are weighed into a 2-liter four-necked flask, and, using 0.5 g tin oxalate and 0.25 g H3PO3 with a simultaneous N₂ stream, heated to 240° C. Stirring is continued in these conditions until AN<3. Then the mixture is cooled, filtered and filled.

Refractive index 20° C.: 1.4518

Hazen color units: 33

AN (acid number): 0.5 mg KOH/g

GC distribution (areas-%):

| | |
|---|---|
| free glycerol | 5.3% |
| monoglycerides | 34.8% |
| diglycerides | 46.4% |
| triglycerides | 13.5% |

### Example 2

339 g (3.6848 mol) of glycerol and 1161 g (7.3677 mol) of precursor fatty acid are weighed into a 2-liter four-necked flask and, using 1.5 g tin oxalate and 0.75 g H3PO3 and a simultaneous N₂ stream, are heated to 240° C. Stirring is continued in these conditions until AN<3. Then the mixture is cooled and filled.

Characteristics:

OH# (hydroxyl number): 134 mg KOH/g

S.N. (saponification number): 315 mg KOH/g

AN: 2.5 mg KOH/g

Refractive index 20° C.: 1.4504

Hazen color units: 169

### Example 3

94.7g (1.0293 mol) of glycerol and 405.3 g (2.5720 mol) of precursor fatty acid are weighed into a 1-liter four-necked flask, and using 0.5 g tin oxalate and 0.25 g H3PO3 and a simultaneous N₂ stream, are heated to 240° C. Stirring is continued in these conditions until AN<1. Then the mixture is cooled and filled.

Refractive index 20° C.: 1.4496

Hazen color units: 32

AN: 0.6 mg KOH/g

### Example 4

141.8 g (1.5413 mol) of glycerol and 358.2 g (3.0836 mol) of 2-ethylbutyric acid are weighed into a 1-liter four-necked flask and, with a simultaneous N₂ stream, are heated to 240° C. Stirring is continued in these conditions until AN<1.

Then the mixture is cooled and filled.

Refractive index 20° C.: 1.5242

Hazen color units: 130

AN: 0.3 mg KOH/g

### Example 5

139.2 g (1.5130 mol) of glycerol, 175.9 g (1.5143 mol) of 2-ethylbutyric acid and 184.9 g (1.5141 mol) of benzoic acid are weighed into a 1-liter four-necked flask and, under an N₂ stream, are heated to 240° C. Stirring is continued in these conditions until AN<1, Then the mixture is cooled, worked up and filled.

Refractive index 20° C.: 1.5002

Hazen color units: 106

AN: 0.3 mg KOH/g

### Example 6

58.2 g (0,6326 mol) of glycerol, 127 g (0.6326 mol) of lauric acid and 115.9 g (0.9491 mol) of benzoic acid are weighed into a 500-ml four-necked flask and, under an N₂ stream, are heated to 240° C. Stirring is continued in these conditions until AN<1. Then the mixture is cooled and filled.

Characteristics: OH#: 64 mg KOH/g

S.N.: 321 mg KOH/g

AN: 1.0 mg KOH/g

### Example 7

65.9 g (0.7163 mol) of glycerol, 102.8 g (0.7164 mol) of octancic acid and 131.3 g (1.0752 mol) of benzoic acid are weighed into a 500-ml four-necked flask and, under an N₇ stream, are heated to 240° C. Stirring is continued in these conditions until AN<2. Then the mixture is cooled and filled.

Characteristics: OH#: 76 mg KOH/g

S.N.: 372 mg KOH/g

AN: 1.2 mg KOH/g

Refractive index 20° C.: 1.5092

### Example 8

67.6 g (0.7348 mol) of glycerol and 232.4 g (1.4687 mol) of isononanoic acid are weighed into a 500-ml four-necked flask and, with a simultaneous N₂ stream, are heated to 240° C. Stirring is continued in these conditions until AN<1.

Then the mixture is cooled and filled.

Refractive index 20° C.: 1.4498

Hazen color units: 13

AN: 0.6 mg KOH/g

### Example 9

149.3 g (1.1127 mol) of trimethylolpropane and 350.7 g (2.2255 mol) of precursor fatty acid are weighed into a 1-liter four-necked flask and, with a simultaneous N₂ stream, are heated to 240° C. Stirring is continued in these conditions until AN<1. Then the mixture is cooled and filled.

Refractive index 20° C.: 1.452

Hazen color units: 183

AN: 1.0 mg KOH/g

### Example 10

111.8 g (0.8212 mol) of pentaerythritol and 388.2 g (2.4635 mol) of precursor fatty acid are weighed into a 1-liter four-necked flask and, with a simultaneous N₂ stream, are heated to 240° C. Stirring is continued in these conditions until AN<1. Then the mixture is cooled and filled.

Hazen color units: 132

AN: 0.5 mg KOH/g

### Example 11

77.2 g (0.8391 mol) of glycerol, 120.4 g (0.8391 mol) of caprylic acid and 102.4 g (0.8385 mol) of benzoic acid are weighed into a 500-ml four-necked flask and, under an N₂ stream, are heated to 240° C. Stirring is continued in these conditions until AN<2. Then the mixture is cooled and filled.

Characteristics: OH#: 172 mg KOH/g

S.N.: 347 mg KOH/g

AN: 1.4 mg KOH/g

### Example 12

### Penetration of Active Substances and Formulation Compatibility

To investigate the penetration behavior of different cosmetic active substances from cosmetic formulations that contained the polyol partial esters according to the invention, penetration tests were carried out with prepared pig skin in modified Franz diffusion cells. The chemically untreated pig skin used was obtained from the Bio-Abattoir Thönes Natur Verbund Wachtendonk. These penetration studies are based on the reference method OECD Guideline TG 428 (Skin absorption: in vitro Method). Retinol, alpha-tocopherol and phytosphingosine salicylate were used as model active substances.

### Method:

The skin undergoes quality control by means of TEWL (measurement of transepidermal water loss), which should be between 10 and 30 g/m² h. The prepared pig skin strips are stored at −20° C. for max. 4 months. For the penetration test, the bristles and the layer of fat are removed from the thawed pig skins. Then a 1 mm thick strip of skin is taken with a dermatome. Circular pieces with a diameter of 1.5 cm are punched from this strip of skin, and are stretched over the Franz cells. Then the test substance (20-40 mg/cm²) is distributed on the skin. The Franz diffusion cells were left in the climatic chamber for 24 hours at a temperature of 32° C. and an air humidity of 50%. By stirring continuously at 150 rev/min with a magnetic stirrer, the acceptor fluid (PBS buffer) is kept homogeneous and the underside of the skin is constantly rinsed. Then any cream still present was removed with a cotton bud and was dissolved in 1 ml methanol. The pig skin was cut into small pieces and was put in 5 ml methanol, and the emollient was extracted from the skin for 24 h on a shaker at 300 rev/min. As control, in each case one untreated pig skin, and three pig skins were treated with 30μL of a 1% caffeine solution and determined. Caffeine serves in this case as control for the permeability of the pig skin. If the amount of caffeine found in the receptor medium is not between 1 and 10% of the amount applied, the skin is not to be used for penetration tests of this type.

### Standard Emulsion: see Table 2

TABLE 2
Standard emulsion

| Ingredients | wt % |
|---|---|
| Ceteareth-25 | 2.0 |
| Bis-PEG/PPG-16/16 PEG/PPG-16/16 dimethicone; | 1.0 |
| caprylic/capric triglyceride¹⁾ | |
| Cetearyl alcohol | 5.0 |
| Stearic acid | 0.5 |
| Polyol partial esters | 15.0 |
| Test active substance (retinol or tocopherol | 0.5 |
| or phytosphingosine salicylate³⁾ | |
| Water | 72.65 |
| Glycerin | 3.0 |
| Carbomer | 0.15 |
| Preservative² | 0.2 |
| Sodium hydroxide solution 10% | to pH 5.5 |

¹ABIL ® Care 85 (Evonik Goldschmidt GmbH)
²EUXYL ® K 220 (Schülke)
³PHYTOSPHINGOSINE SLC (Evonik Goldschmidt GmbH)

### Sample Preparation for HPLC Analysis:

### Determination of the Content of Active Substance in the Emulsion

For this, 0.5-1 g of the emulsion and the same amount of sodium sulfate are transferred to a sample bottle. Then 20 ml methanol is added. This solution is shaken on an orbital shaker at 300 rev/min. After 24 hours, 1.5 ml of the suspension is put in an Eppendorf tube and centrifuged in a centrifuge at 13000 rev/min for 3 min. The clear supernatant is pipetted into an HPLC sample vessel and measured by HPLC.

### Determination of the Content of Active Substance in the Supernatant:

The supernatant is washed from the skin with a cotton bud and transferred to an Eppendorf tube filled with 1 ml methanol. The Eppendorf tube is shaken at 1400 rev/min with a tube shaker. After 30 minutes these Eppendorf tubes are centrifuged with a centrifuge at 13000 rev/min for 3 min. The clear supernatant is pipetted into an HPLC sample vessel and measured by HPLC.

### Determination of the Content of Active Substance in the Skin:

The skin is cut into small pieces with scissors and put in a 15-ml sample bottle. Then 5 ml methanol is added. This sample bottle is shaken on an orbital shaker at 300 rev/min. After 24 hours, 1.5 ml of the suspension is out in an Eppendorf tube and centrifuged in a centrifuge at 13000 rev/min for 3 min. The clear supernatant is pipetted into an HPLC sample vessel and measured by HPLC.

Determination of the Content of Active Substance from the Receptor:

The receptor medium is pipetted into an Eppendorf tube and centrifuged in a centrifuge at 13000 rev/min for 3 min. The clear supernatant is transferred to an HPLC sample vessel and measured by HPLC.

### HPLC Conditions for Retinol and Tocopherol:

Column: PerfectSil Target ODS-3 HD 4.6×150 mm HPLC column (MZ Analysentechniken GmbH, Mainz, Germany)

Eluent: isocratic with methanol

Flow: 1 ml/min

Injection volume: 10 μL

UV wavelength for retinol: 272 nm or 325 nm

Fluorescence wavelengths: excitation at 295 nm and emission at 330 nm

### HPLC Conditions for Phytosphingosine Salicylates:

Column: Kromasil C18 4.6×250 mm HPLC column (MZ Analysentechniken GmbH, Mainz, Germany)

Fluent: isocratic with methanol/water (90/10 v/v)

Flow: 1 ml/min

Injection volume: 10 μL

UV wavelength for phytosphingosine salicylates: 300 nm

### HPLC Conditions:

Column: PerfectSil Target ODS-3 HD 4.6×150 mm HPLC column (MZ Analysentechniken GmbH, Mainz, Germany)

Eluent: isocratic with methanol

Flow: 1 ml/min

Injection volume: 10 μL

UV wavelength for retinol: 272 nm or 325 nm

Fluorescence wavelengths: excitation at 295 nm and emission at 330 nm

### Results:

The penetration results shown in the following tables are the mean values from determinations carried out six times. The active substance was not detected in the receptor medium in any of the tests. The active substance was either in the skin or on the surface of the skin, i.e. in the supernatant that was washed away.

The C8/C10 esters of glycerol were selected as substances for an informative comparison series. The (not according to the invention) triester of glycerol⁴⁾ and the (not according to the invention) partial ester of glycerol with octanoic acid (proportion of monoglyceride >80%)⁵⁾ served as known end points. This reference product is known to intensify the penetration of active substances, but it is a solid with very high polarity, with very limited applicability in cosmetic formulations owing to restricted compatibility with common cosmetic oils. In contrast, the examples according to the invention are all clear liquids with very good compatibility with common cosmetic oils.

TABLE 3
Penetration results for phytosphingosine salicylates.

| Emollient/ | Active | Active | Active |
|---|---|---|---|
| (amount of C8/C10 | substance | substance | substance |
| acid used for | in the | in the | in the |
| synthesis) | supernatant % | skin % | receptor % |
| Caprylic/capric | 82.4 | 17.7 | 0 |
| triglyceride⁴⁾ | | | |
| (3 mol) | | | |
| Example 3 | 41.4 | 58.6 | 0 |
| (2.5 mol) | | | |
| Example 2 | 29.1 | 70.9 | 0 |
| (2.0 mol) | | | |
| Example 1 | 26.8 | 73.2 | 0 |
| (1.5 mol) | | | |
| Glyceryl | 26.7 | 73.3 | 0 |
| monocaprylate⁵⁾ | | | |
| (1 mol) | | | |

⁴⁾TEGOSOFT ® CT (Evonik Goldschmidt GmbH)
⁵⁾IMWITOR ® 308 (Sasol)

It is clear from Table 3 that the polyol partial esters according to the invention bring about greatly increased penetration of the active substance into the skin compared to the full ester caprylic/caoric triglyceride. In particular the values for examples 1 and 2 according to the invention have an equally good penetration level as when using the known penetration intensifier glyceryl monocaprylate.

The advantage of the polyol partial esters according to the invention, compared to the polyol partial ester with proportion of monoglyceride >80%, is the greatly improved compatibility with common cosmetic oils (Table 4).

TABLE 4
Solubility comparison of a polyol partial ester
according to the invention compared to glyceryl
monocaprylate in various common cosmetic emollients.

Solubility in wt % in the
test emollients

| | | Glyceryl mono- |
|---|---|---|
| Test emollients | Example 1 | caprylate⁵⁾ |
| Mineral oil | >75% | <5% |
| Diethylhexyl | >75% | at 75% solid, |
| carbonate | | solidified mass |
| Octyldodecanol | >75% | at 75% solid, |
| | | solidified mass |
| Isohexadecane | >75% | at 75% solid, |
| | | solidified mass |
| Oleyl erucate | >75% | <50% |

It is clear from the solubility values in Table 4 that the polyol partial esters according to the invention show far better compatibility with common cosmetic oils. Based on this wider range of usability, easier handling (liquid products), more economical manufacture (no high-cost purification required), with comparable penetration intensifying action, they are far superior to the products of the prior art.

Tables 5 and 6 summarize the results of penetration tests with the model active substances retinol and tocopherol. Once again, the intensifying action of polyol partial esters according to the invention on penetration of active substances into the skin can clearly be seen.

TABLE 5
Penetration results for retinol.

| Emollient/ | Active | Active | Active |
|---|---|---|---|
| (amount of C8/C10 | substance | substance | substance |
| acid used for | in the | in the | in the |
| synthesis) | supernatant % | skin % | receptor % |
| Caprylic/capric | 69.2 | 30.8 | 0 |
| triglyceride⁴⁾ | | | |
| (3 mol) | | | |
| Example 3 | 38.9 | 61.1 | 0 |
| (2.5 mol) | | | |
| Example 2 | 29.8 | 70.2 | 0 |
| (2.0 mol) | | | |
| Example 1 | 30.8 | 69.2 | 0 |
| (1.5 mol) | | | |

TABLE 6
Penetration results for tocopherol.

| Emollient/ | Active | Active | Active |
|---|---|---|---|
| (amount of C8/C10 | substance | substance | substance |
| acid used for | in the | in the | in the |
| synthesis) | supernatant % | skin % | receptor % |
| Caprylic/capric | 62.9 | 37.1 | 0 |
| triglyceride⁴⁾ | | | |
| (3 mol) | | | |
| Example 3 | 35.7 | 64.3 | 0 |
| (2.5 mol) | | | |
| Example 2 | 25.8 | 74.2 | 0 |
| (2.0 mol) | | | |
| Example 1 | 27.0 | 73.0 | 0 |
| (1.5 mol) | | | |

### Example 13

### UV Filter Solubility

The very good UV filter solubility of the polyol partial esters according to the invention can clearly be demonstrated by solubility tests of UV filters in the oils according to the invention.

The two UVB filters ethylhexyl triazone (Uvinul® T 150 (BASF SE)) and benzophenone-3 (Uvinul® M 40 (BASF SE)) were used as filter substances.

For determining the dissolving power for these three UV filters, in each case a specified amount (50 g) of one of the compounds according to the invention or of the comparative oils was preheated to 22° C. 1 wt % of a UV filter was added and stirred, until this amount had dissolved completely and homogeneously. This operation was repeated until the maximum soluble amount of the UV filter was exceeded. At higher concentrations, often a longer stirring time of several hours is necessary for complete dissolution.

When in this way the maximum concentration had been determined approximately, for fine determination the concentration range was repeated around this maximum concentration with smaller initial weighed amounts of the UV filter.

TABLE 7
UV filter solubilities in polyol partial esters
according to the invention and comparative oils.

| | Ethylhexyl | |
|---|---|---|
| Emollient | triazone | Benzophenone-3 |
| Example 1 | 14.1% | 18.2% |
| Example 2 | 16.0% | 15.7% |
| Example 3 | 11.6% | 17.2% |
| Example 4 | 8.8% | 18.7% |
| Example 5 | 7.8% | 23.1% |
| Example 6 | 8.5% | 21.4% |
| Example 7 | 9.0% | 25.5% |
| Example 8 | 9.1% | 12.3% |
| C12-15 alkyl | 8.0% | 12.0% |
| benzoate | | |
| Isopropyl | 6.0% | 9.0% |
| palmitate | | |
| Ethylhexyl | 4.0% | 7.0% |
| palmitate | | |
| Isononyl | 3.0% | 8.0% |
| isononanoate | | |
| Diethylhexyl | 7.0% | 10.0% |
| carbonate | | |
| PPG-15 stearyl | 7.0% | 9.0% |
| ether | | |

It is clear from the percentage solubilities of the UV filters that the polyol partial esters according to the invention display far better UV filter solubilities than conventional emollients. Even C12-15 alkyl benzoate, which is used, owing to its excellent filter solubility, in almost all sunscreen products, is surpassed.

### Example 14

### Intensification of the SEE (Sun Protection Factor)

To verify the effects of the better filter solubility on intensification of the SPF, the substances from example 4 and example 11 were in each case used at 8% in an O/W sunscreen lotion and the in-vitro SPF was determined with the Optometrics SPF 290 S analyzer. Lotions with the standard emollients diethylhexyl carbonate/C12-15 alkyl benzoate in the weight ratio 1/1 and caprylic/capric triglyceride were used as comparative preparations.

| | Example formulation | | | |
|---|---|---|---|---|
| | I | II | V1 | V2 |
| Cetearyl alcohol | 2.0% | 2.0% | 2.0% | 2.0% |
| Diethylhexyl carbonate | | | 4.0% | |
| C12-15 alkyl benzoate | | | 4.0% | |
| Caprylic/capric | | | | 8.0% |
| triglyceride | | | | |
| Example 4 | 8.0% | | | |
| Example 11 | | 8.0% | | |
| Tocopheryl acetate | 0.5% | 0.5% | 0.5% | 0.5% |
| Octocrylene | 4.2% | 4.2% | 4.2% | 4.2% |
| Ethylhexyl triazone | 4.4% | 4.4% | 4.4% | 4.4% |
| Butyl | 4.2% | 4.2% | 4.2% | 4.2% |
| methoxydibenzoylmethane | | | | |
| Titanium dioxide, | 5.0% | 5.0% | 5.0% | 5.0% |
| trimethoxycaprylyl | | | | |
| silane⁶⁾ | | | | |
| Xanthan gum | 0.2% | 0.2% | 0.2% | |
| Carbomer⁷⁾ | | | 0.1% | |
| Ethylhexyl stearate | | | 0.4% | |
| Cetearyl glucoside⁸⁾ | 2.0% | 2.0% | 2.0% | 2.0% |
| Xanthan gum | 0.4% | 0.4% | | 0.6% |
| Water | 67.9% | 67.9% | 67.2% | 67.9% |
| Preservative | 1.0% | 1.0% | 1.0% | 1.0% |
| Euxyl K 300 | | | | |
| Perfume Sunea | 0.2% | 0.2% | 0.2% | 0.2% |
| Sodium hydroxide 10% | | | 0.6% | |
| SPF BASF Sunscreen | 40 | 40 | 40 | 40 |
| Simulator (calculated) | | | | |
| SPF Optometrics | 75 | 54 | 40 | 42 |
| 0.75 mg/cm², Schönberg | | | | |
| PMMA plates | | | | |

⁶⁾TEGO ® Sun T 805 (Evonik Goldschmidt GmbH)
⁷⁾TEGO ® Carbomer 141 (Evonik Goldschmidt GmbH)
⁸⁾TEGO ® Care CG 90 (Evonik Goldschmidt GmbH)

The measured in vitro SPF values of examples I and II according to the invention clearly show that the polyol partial esters according to the invention give far better sun protection factors than the conventional emollients C12-15 alkyl benzoate and caprylic/capric triglyceride in comparative examples V1 and V2. This is also clear from the fact that the theoretically calculated SPF value is reached exactly with the latter, whereas with the polyol partial esters according to the invention it is surpassed by 35% or 85% respectively.

### Examples of Use:

The cosmetic emulsions described should provide an example for illustrating the usability of the polyol partial esters according to the invention in cosmetic emulsions.

Preparation was carried out in each case by introducing the aqueous phase into the out phase and then homogenizing by the usual methods.

### O/W Creams

| | Example formulation | | | | |
|---|---|---|---|---|---|
| | 1 | 2 | 3 | 4 | 5 |
| Polyglyceryl-3 | 3.0% | | 2.0% | | 2.0% |
| methylglucose | | | | | |
| stearate⁹⁾ | | | | | |
| Polyglyceryl-3 | | 3.0% | | 2.8% | |
| stearate/citrate¹⁰⁾ | | | | | |
| Glyceryl stearate | 2.0% | 2.0% | 2.0% | 2.0% | 3.5% |
| Stearyl alcohol | 1.0% | 2.0% | 1.0% | 2.0% | 1.5% |
| Distearyldimonium | | | 1.0% | | |
| chloride | | | | | |
| Sodium cetearyl | | | | 0.2% | |
| sulfate | | | | | |
| Bis-PEG/PPG-20/5 | | | | | 1.0% |
| PEG/PPG-20/5 | | | | | |
| dimethicone, methoxy | | | | | |
| PEG/PPG-25/4 | | | | | |
| dimethicone; | | | | | |
| caprylic/capric | | | | | |
| triglyceride¹¹⁾ | | | | | |
| Isopropyl | 5.3% | | | 10.0% | |
| palmitate | | | | | |
| Example 2 | | 4.0% | | | |
| Example 4 | 5.0% | | | | |
| Caprylic/capric | | 3.5% | 10.0% | 5.0% | |
| triglyceride | | | | | |
| Almond oil | | | | 5.0% | |
| Avocado oil | | 5.0% | | | |
| Diethylhexyl | 9.5% | | | | 8.5% |
| carbonate | | | | | |
| Ethylhexyl | | | 9.0% | | 5.0% |
| palmitate | | | | | |
| Decyl cocoate | | 7.0% | | | |
| Dimethicone | | | | | 2.0% |
| Ceramide III | | | 0.1% | | |
| Salicyloyl phyto- | | | | 0.1% | |
| sphingosine | | | | | |
| Caprylic/capric | | | | | 0.2% |
| triglyceride; | | | | | |
| xymenynic acid¹²⁾ | | | | | |
| Cetyl ricinoleate | | | | | 1.0% |
| Glycerin | 3.0% | 3.0% | 6.0% | 3.0% | |
| Tetrapeptide-17, | 2.5% | | | | |
| glycerin, butylene | | | | | |
| glycol, aqua¹³⁾ | | | | | |
| Curcuma longa | | 0.5% | | | |
| (turmeric root | | | | | |
| extract)¹⁴⁾ | | | | | |
| Carbomer | 0.2% | | | | |
| Demineralized | to 100% | to 100% | to 100% | to 100% | to 100% |
| water | | | | | |
| Benzyl alcohol, | | 1.0% | | 1.0% | |
| benzoic acid, | | | | | |
| sorbic acid¹⁵⁾ | | | | | |
| NAOH (10% solution) | | | | | |
| adjustment of pH | | q.s. | | q.s. | |
| to 5.0) | | | | | |
| Methylisothiazolinone, | 0.8% | | | | 0.8% |
| methylparaben, | | | | | |
| ethylparaben; | | | | | |
| dipropylene glycol¹⁶⁾ | | | | | |
| Phenoxyethanol, | | | 0.7% | | |
| ethylhexylglycerol¹⁷⁾ | | | | | |

⁹⁾TEGO ® Care 450 (Evonik Goldschmidt GmbH)
¹⁰⁾TEGO ® Care PSC 3 (Evonik Goldschmidt GmbH)
¹¹⁾ABIL ® Care XL 80 (Evonik Goldschmidt)
¹²⁾TEGO ® Xymenynic (Evonik Goldschmidt)
¹³⁾TEGO ® Pep 4-17 (Evonik Goldschmidt)
¹⁴⁾TEGO ® Tumerone (Evonik Goldschmidt)
¹⁵⁾Rokonsal ® BSB-N (ISP)
¹⁶⁾Microcare ® MEM (Thor)
¹⁷⁾Euxyl ® PE 9010 (Schülke)

### O/W Sunscreen Lotion:

| | Example |
|---|---|
| | formulation |
| | 6 |
| Polyglyceryl-3 methylglucose distearate⁹⁾ | 3.0% |
| Glyceryl stearate | 0.5% |
| Stearyl alcohol | 0.5% |
| Diethylhexyl carbonate | 3.0% |
| Caprylic/capric triglyceride | 2.0% |
| Example 2 | 5.0% |
| Octocrylene | 2.0% |
| bis-Ethylhexyloxyphenol methoxyphenyl | 3.0% |
| triazine | |
| Polysilicone-15 | 2.0% |
| Titanium dioxide; trimethoxycaprylylsilane | 5.0% |
| Glycerin | 3.0% |
| Xanthan gum | 0.2% |
| Carbomer | 0.3% |
| Demineralized water | to 100% |
| NaOH (5% solution) (adjustment of pH to 6.0) | q.s. |
| Methylisothiazolinone, methylparaben, | 0.8% |
| ethylparaben; dipropylene glycol¹⁶⁾ | |

### O/W Self-Tanning Lotion:

| | Example |
|---|---|
| | formulation |
| | 7 |
| Polyglyceryl-3 stearate/citrate¹⁰⁾ | 3.0% |
| Ceteareth-25 | 0.5% |
| Glyceryl stearate | 2.5% |
| Stearyl alcohol | 1.0% |
| Isopropyl palmitate | 3.0% |
| Example 2 | 3.0% |
| Mineral oil | 7.0% |
| Jojoba oil | 3.0% |
| Glycerin | 3.0% |
| Dihydroxyacetone | 5.0% |
| Demineralized water | to 100% |
| Citric acid (10% solution) | q.s. |
| (adjustment of pH to 4.0) | |
| Methylisothiazolinone, methylparaben, | 0.8% |
| ethylparaben; dipropylene glycol¹⁶⁾ | |

O/W Hair Cream with UV Protection:

| | Example |
|---|---|
| | formulation |
| | 8 |
| Polyglyceryl-3 methylglucose distearate⁹⁾ | 3.0% |
| Example 7 | 4.0% |
| Apricot kernel oil | 2.5% |
| Almond oil | 2.5% |
| Cetearyl ricinoleate | 1.0% |
| Isopropyl myristate | 2.0% |
| Cetearyl alcohol | 1.0% |
| Glyceryl stearate | 1.5% |
| Glycerin | 3.0% |
| Ethylhexyl methoxycinnamate | 2.0% |
| Polysilicone-19 | 0.5% |
| Silicone quaternium-22 | 0.2% |
| Demineralized water | to 100% |
| Methylisothiazolinone, methylparaben, | 0.8% |
| ethylparaben; dipropylene glycol¹⁶⁾ | |

### W/O—Sunscreen Formulation

| | Example |
|---|---|
| | formulation |
| | 9 |
| Cetyl PEG/PPG-10/1 | 3.0% |
| dimethicone¹⁸⁾ | |
| Cetyl dimethicone | 0.5% |
| Diethylhexyl carbonate | 11.5% |
| C12-15 alkyl benzoate | 3.0% |
| Example 7 | 3.0% |
| Ethylhexyl methoxycinnamate | 7.5% |
| Isoamyl p-methoxycinnamate | 7.5% |
| Ethylhexyl triazone | 1.0% |
| bis-Ethylhexyloxyphenol | 2.5% |
| methoxyphenyl triazine | |
| Titanium dioxide; trimethoxy- | 4.0% |
| caprylylsilane⁶⁾ | |
| Cyclopentasiloxane; | 3.0% |
| dimethicone crosspolymer¹⁹⁾ | |
| Hydrogenated castor oil | 0.5% |
| Microcrystalline wax | 0.5% |
| Glycerin | 3.0% |
| Sodium chloride | 0.8% |
| Creatine | 0.2% |
| Water | to 100% |
| Preservative, perfume | q.s. |

¹⁸⁾ABIL EM 90 (Evonik Goldschmidt GmbH)
¹⁹⁾Dow Corning 9040 Silicone Elastomer Blend (Dow Corning)

### W/O—Makeup

| | Example |
|---|---|
| | formulation |
| | 10 |
| Cetyl PEG/PPG-10/1 | 2.5% |
| dimethicone¹⁸⁾ | |
| Polyglyceryl-4 diiso | 2.5% |
| stearate/polyhydroxy | |
| stearate/sebacate²⁰⁾ | |
| Cetyl dimethicone | 1.0% |
| Diethylhexyl carbonate | 4.5% |
| Ethylhexyl palmitate | 1.5% |
| Example 1 | 2.0% |
| Dimethicone | 3.0% |
| Cyclopentasiloxane | 9.0% |
| Phenyl trimethicone | 1.0% |
| Lauryl dimethicone/polyglyceryl- | 2.0% |
| 3 crosspolymer; | |
| triethylhexanoin²¹⁾ | |
| Nylon-12 | 1.0% |
| Iron oxides | 2.0% |
| Titanium oxide | 6.0% |
| Zinc oxide | 0.5% |
| Glycerin | 3.0% |
| Sodium chloride | 0.8% |
| Creatine | 0.2% |
| Water | to 100% |
| Preservative, perfume | q.s. |

²⁰⁾ISOLAN ® GPS (Evonik Goldschmidt GmbH)
²¹⁾KSG-830 (Shin Etsu)

1. A polyol partial ester consisting of esterification products of at least one polyol, selected from polyols with 3 to 6 carbon atoms containing 2 to 6 OH groups with at least one carboxylic acid containing 5 to 18 carbon atoms, with the proviso that the molar ratio of the OH groups in the polyol to the acyl groups of the carboxylic acids in the polyol partial ester is between 1:0.90 and 1:0.35.

2. The polyol partial ester as claimed in claim 1, wherein at 1 bar pressure it has a melting point below 22° C.

3. The polyol partial ester as claimed in claim 1, wherein each individual polyol ester of a particular degree of esterification does not account for more than 80 wt % of the total polyol partial ester.

4. The polyol partial ester as claimed in claim 1, wherein the content of monoester relative to the total polyol partial ester is less than 60 wt %,

5. The polyol partial ester as claimed in claim 1, wherein the at least one carboxylic acid, which is present in the polyol partial ester in esterified form, is selected from the group consisting of neopentanoic acid, isoamylic acid, pentanoic acid, n-hexanoic acid, 2-ethylbutanoic acid, cyclohexanecarboxylic acid, n-octanoic acid, 2-ethylhexanoic acid, isononanoic acid, 3,5,5-trimethylhexanoic acid, n-decanoic acid, iso-decanoic acid, lauric acid and 2-butyloctanoic acid.

6. The polyol partial ester as claimed in claim 1, wherein the polyol glycerol, in which at least one carboxylic acid is a mixture consisting of n-octanoic acid and n-decanoic acid in a weight ratio from 40:80 to 20:60 and, per mol of OH group contained in the polyol, a total of 0.45 to 0.70 mol acyl groups of the carboxylic acid are present in esterified form in the polyol partial ester.

7. A cosmetic or pharmaceutical formulation comprising a polyol partial ester as claimed in claim 1 and at least one cosmetic and/or pharmaceutical active substance.

8. The formulation as claimed in claim 7, comprising
(a) 0.1-60 wt %, of polyol partial esters according to the invention,
(b) 0.001-15 wt %, of at least one cosmetic and/or pharmaceutical active substance,
(c) 0.1-20 wt %, of emulsifiers and/or coemulsifiers,
(d) 0.1-40 wt %, of other oily substances,
(e) 0-99 wt %, water and optionally usual excipients and additives.

9. The cosmetic formulation as claimed in claim 7, wherein said cosmetic formulation is a sunscreen formulation and comprises 0.01-15 wt % UV filters relative to the total formulation.

10. The formulation as claimed in claim 9, comprising
(a) 0.1-60 wt % of polyol partial esters,
(b) 0.01-20 wt % of at least one UV filter substance,
(c) 0.1-20 wt % of emulsifiers and/or coemulsifiers,
(d) 0.1-40 wt % of other oily substances,
(e) 0-99 wt % water and optionally usual excipients and additives.

11. A method of producing the cosmetic sunscreen formulation as claimed in claim 9 comprising the process steps
(A) dissolving the organic UV filters in an oily phase containing polyol partial esters,
(B) providing an aqueous phase and combining the aqueous and oily phase,
(C) homogenizing and, in the case of a hot process, cooling.

12-14. (canceled)

15. The formulation as claimed in claim 8, comprising:
(a) 1-25 wt % of polyol partial esters,
(b) 0.05-10 wt % of at least one cosmetic and/or pharmaceutical active substance,
(c) 0.2-10 wt % of emulsifiers and/or coemulsifiers,
(d) 0.2-20 wt % of other only substances, and
(e) 10-95 wt % water and optionally usual excipients and additives

16. The formulation as claimed in claim 8, comprising:
(a) 3 to 15 wt % of polyol partial esters,
(b) 0.1 - 5 wt % of at least one cosmetic and/or pharmaceutical active substance,
(c) 0.5-6 wt % of emulsifiers and/or coemulsifiers,
(d) 0.5-15 wt % of other only substances, and
(e) 30-75 wt % water and optionally usual excipients and additives.

17. The formulation as claimed in claim 10, comprising:
(a) 1 to 25 wt % of polyol partial esters,
(b) 0.05 - 15 wt % of at least one UV filter substance,
(c) 0.2-10 wt % of emulsifiers and/or coemulsifiers,
(d) 0.2-20 wt % of other only substances, and
(e) 10-95 wt % water and optionally usual excipients and additives.

18. The formulation as claimed in claim 10, comprising:
(a) 3 to 15 wt % of polyol partial esters,
(b) 0,1 - 10 wt % of at least one UV filter substance,
(c) 0.5-6 wt % of emulsifiers and/or coemulsifiers,
(d) 0.5-15 wt % of other only substances, and
(e) 30-75 wt % water and optionally usual excipients and additives.
